(19) United States
(12) Patent Application Publication
(10) Pub. No.: US 2023/0310570 A1
(43) Pub. Date: Oct. 5, 2023

(54) COMPOSITIONS AND METHODS FOR VACCINATION OF JUVENILES AGAINSTRESPIRATORY SYNCYTIAL VIRUS INFECTION

(71) Applicants: UNIVERSITY OF PITTSBURGH-OF THE COMMONWEALTH SYSTEM OF HIGHER EDUCATION, Pittsburgh, PA (US); CALDER BIOSCIENCES INC., New York, NY (US)

(72) Inventors: Jessica L. Kosanovich, Cranberry Township, PA (US); Katherine M. Eichinger, Pittsburgh, PA (US); Kerry M. Empey, Mars, PA (US); Mark A. Yondola, Medford, NY (US); Christopher P. Marshall, New York, NY (US)

(21) Appl. No.: 18/042,093

(22) PCT Filed: Aug. 17, 2021
(86) PCT No.: PCT/US2021/046324

§ 371 Date: Feb. 17, 2023

Related U.S. Application Data
(60) Provisional application No. 63/066,627, filed on Aug. 17, 2020.

(51) Int. Cl. A61P 31/14; A61K 39/12; C07K 14/005; A61K 39/39

(52) U.S. Cl. CPC A61K 39/12; A61K 39/39; A61P 31/14; C07K 14/005; A61K 2039/55

(57) ABSTRACT

The present invention provides methods useful for vaccination against respiratory syncytial virus (RSV), including vaccination of juvenile subjects. In some embodiments such methods involve administration of an RSV F polypeptide stabilized in a prefusion conformation and a Th-balanced adjuvant to a juvenile subject. The present invention also provides a variety of compositions useful in such methods.

### CROSS-REFERENCE TO RELATED APPLICATIONS

This application is a National Stage of International Application No. PCT/US2021/046324, filed on Aug. 17, 2021, which claims the benefit of priority of U.S. Provisional Pat. Application No. 63/066,627 filed on Aug. 17, 2020, the content of which is hereby incorporated by reference in its entirety.

### SEQUENCE LISTING

The instant application contains a Sequence Listing which has been submitted electronically in ASCII format and is hereby incorporated by reference in its entirety. Said ASCII copy, created on Aug. 13, 2021, is named Calder_011_WO1_SL.txt and is 43,480 bytes in size.

### STATEMENT OF GOVERNMENT SUPPORT

This invention was made with government support under grant numbers AI140941 and AI112124 awarded by the National Institutes of Health. The government has certain rights in the invention.

### FIELD OF THE INVENTION

The disclosure generally relates to vaccination against respiratory syncytial virus (RSV). The disclosure includes various compositions and methods for vaccination, including various compositions and methods for vaccination of juvenile human subjects.

### BACKGROUND OF THE INVENTION

Respiratory syncytial virus (RSV) is estimated to cause 30 million acute respiratory tract infections each year, resulting in an estimated 3.2 million annual hospitalizations worldwide and approximately 60,000 in-hospital deaths in children less than 5 years old (Shi et al., Lancet, 2017;390(10098):946-58; Jha et al., Wellcome Trust-Funded Monographs and Book Chapters, Sheffield UK2016). Infants less than 6 months of age account for nearly 50% of all RSV-related hospital admissions and in-hospital deaths (Shi et al., Lancet, 2017;390(10098):946-58), highlighting the need for a vaccine able to provide RSV protection early in life. Given the strong association between severe RSV infection during infancy and the subsequent development of asthma and impaired lung function, prevention of acute, RSV-related disease may also have long-term beneficial effects (Zomer-Kooijker et al., PLoS One, 2014;9(1):e87162; Feldman et al., Am. J. Respir. Crit. Care Med., 2015;191(1):34-44).

The development of an effective RSV vaccine for protection of infants has been hampered by neonatal immune immaturity (PrabhuDas et al., Nat. Immunol., 2011;12(3):189-94), the short time frame between birth and first RSV exposure, and the risk of vaccine-enhanced respiratory disease (VERD) in infants that was first identified in the late 1960s when a formalin-inactivated, alum-adjuvanted RSV (FI-RSV) vaccine resulted in the death of two children (Acosta et al., Clin. Vaccine Immunol., 2015;23(3):189-95; Kim et al., Am. J. Epidemiol., 1969;89(4):422-34). The development of enhanced respiratory disease (ERD), including VERD, is believed to be associated with the development of a Th2 skewed immune response. As such, there has been an effort in the field to find ways of protecting infants against RSV infection without vaccinating the infants themselves.

One approach being investigated involves vaccinating pregnant mothers against RSV so that the mothers might then pass immunity to their offspring in a so-called “maternal-to-infant” vaccination approach. See, for example, WO2019/0178521.

Another approach that has been considered but not yet effectively employed is to vaccinate the older siblings of infants - including school-aged children - because, most commonly, it is these school-aged children that bring RSV infection into their home and thereby infect their younger infant siblings. Moreover, there can be serious complications of RSV infection in some school-age children also. However, there has been uncertainty about whether such an approach would be effective because it is known that multiple natural RSV exposures are required for children to mount a full immune response to RSV and it is also known that the protection induced through natural infection is relatively short-lived. Another concern with vaccinating children has been that the existence of pre-existing Th2-skewed immunity to RSV (most children have been exposed to a natural RSV infection by early childhood and are thus RSV-experienced and/or RSV-seropositive) might predispose such children to develop VERD.

The present invention is directed to this previously unproven approach of vaccinating non-infant children against RSV.

### SUMMARY OF THE INVENTION

The present invention is based, in part, on a series of important discoveries that are described in more detail in the Examples sections of this patent specification. In brief, the inventors of the present patent application have discovered that vaccination of RSV-experienced/RSV-seropositive juveniles with a recombinant RSV F antigen stabilized in its pre-fusion conformation (referred to herein as “preF”) both boosted neutralizing antibody production and afforded protection from RSV reinfection in the vaccinated juveniles. Furthermore, the inventors showed that co-administration of a Th1 balanced adjuvant (but not a Th2 skewing adjuvant) was sufficient to prevent/reverse the expected Th2 skewed immune response observed in RSV-experienced/RSV-seropositive juveniles - suggesting that there may be less risk of development of enhanced respiratory disease in subjects vaccinated with a combination of “preF” and a Th-balanced adjuvant.

Building on these discoveries, and other discoveries presented herein, the present invention provides a variety of new and improved compositions and methods for the prevention and/or amelioration of RSV infection by vaccination of juvenile subjects. Importantly, the compositions and methods described herein are protective against RSV infection, elicit a more desirable Th1-balanced immune response, and reduce the risk of juveniles developing vaccine-enhanced respiratory disease (VERD) and eosinophilia.

Accordingly, in some embodiments the present invention provides methods for the prevention or amelioration of RSV infection in juvenile subjects, such methods comprising administering to a juvenile subject an effective amount of both an RSV F polypeptide stabilized in a prefusion conformation and a Th1-balanced adjuvant (or administering an effective amount of a composition comprising both a RSV F polypeptide stabilized in a prefusion conformation and a Th1-balanced adjuvant), thereby preventing or ameliorating RSV infection in the juvenile subject. In some embodiments such methods advantageously reduce the occurrence and/or severity of vaccine-enhanced respiratory disease (VERD) and/or eosinophilia in the subjects. In some embodiments the subjects are human juveniles of school-going age. For example, in some embodiments the subjects are human juveniles of from about 2 to about 12 years of age, from about 2 to about 13 years of age, from about 2 to about 14 years of age, from about 2 to about 15 years of age, from about 3 to about 12 years of age, from about 3 to about 13 years of age, from about 3 to about 14 years of age, from about 3 to about 15 years of age, or from about 4 to about 12 years of age, from about 4 to about 13 years of age, from about 4 to about 14 years of age, from about 4 to about 15 years of age, or from about 5 to about 12 years of age, from about 5 to about 13 years of age, from about 5 to about 14 years of age, from about 5 to about 15 years of age. Similarly, in some embodiments the subjects are human juveniles of about 2, or about 3, or about 4, or about 5, or about 6, or about 7, or about 8, or about 9, or about 10, or about 11, or about 12, or about 13, or about 14, or about 15 years of age. In some embodiments the subjects are RSV-experienced. In some embodiments the subjects are RSV-seropositive.

In other embodiments the present invention provides compositions for use in methods of vaccinating juveniles against RSV or for use in the preparation of an RSV vaccine for administration to juveniles, such compositions comprising: (a) an RSV F polypeptide stabilized in a prefusion conformation and (b) a Th1-balanced adjuvant.

In some embodiments, the RSV F polypeptide used in the methods and/or compositions described herein is stabilized in its pre-fusion trimeric conformation. In some embodiments the RSV F polypeptide comprises a trimerization domain. In some embodiments the RSV F polypeptide comprises a mutation that fills a space within a cavity in a RSV F polypeptide or between RSV F polypeptides. In some embodiments, the RSV F polypeptide comprises a non-natural disulfide bond. In some embodiments, the RSV F polypeptide comprises one or more artificially introduced dityrosine bonds. In some embodiments the RSV F polypeptide is multimerized on a particle or part of a supramolecular complex or incorporated on a VLP.

In some embodiments, the Th1 balanced adjuvant used in the methods and/or compositions described herein comprises a CpG oligonucleotide. In some embodiments, the Th1 balanced adjuvant comprises a CpG oligonucleotide and a delta inulin. In some embodiments, the Th1 balanced adjuvant comprises Advax-SM. In some embodiments, the Th1 balanced adjuvant comprises MPL. In some embodiments, the Th1 balanced adjuvant comprises poly(I:C). In some embodiments, the Th1 balanced adjuvant comprises poly(IC:LC). In some embodiments, the Th1 balanced adjuvant comprises Freunds Complete Adjuvant. In some embodiments, the Th1 balanced adjuvant comprises saponin. In some embodiments, the Th1 balanced adjuvant comprises dQS21. In some embodiments, the Th1 balanced adjuvant comprises an oil-in-water emulsion adjuvant.

These and other aspects of the present invention are described further in the below Detailed Description, Drawings, Examples and Claims sections of this patent application. Furthermore, one of skill in the art will recognize that the various embodiments of the present invention described throughout this patent disclosure can be combined in various different ways, and that such combinations are within the scope of the present invention.

### BRIEF DESCRIPTION OF THE DRAWINGS

FIGS. 1 A-F. Vaccination of RSV-experienced young mice elicits high RSV neutralizing antibody titers and protects against secondary infection. At 5 to 6 days of age, infant BALB/cJ mice were infected with 5x10⁵ PFU/gram of RSV L19. Three weeks later, female mice were immunized with Prefusion RSV F protein (PreF)/Advax-SM, PreF/Alum, or mock-vaccinated with PBS, then boosted 3 weeks after initial immunization. One-week post-boost, mice were bled and subsequently challenged with 5x10⁵ PFU/g of RSV L19. Mice were culled for sample collection at 4- or 8-days post-infection (dpi) (FIG. 1A). RSV neutralizing antibody levels (FIG. 1B), PreF-specific IgG2a (FIG. 1D), PreF-specific IgG1 (FIG. 1E), and IgG2a/IgG1 ratio (FIG. 1F) were obtained from pre-challenge serum. IgG2a/IgG1 ratio was determined by dividing PreF-specific relative IgG2a (µg/mL) by PreF-specific relative IgG1(µg/mL). Left lungs or upper right lungs were harvested to assess viral titers at 4 days post infection () with quantification using H&E plaque assays (FIG. 1C). Viral titers were performed in triplicate and data within each group represent the mean titer for each animal. Data are represented as mean ± SEM. Statistical significance between vaccination groups was calculated using one-way ANOVA with Dunn’s multiple comparison post-test (FIG. 1B and FIG. 1C), one-way ANOVA with Tukey’s multiple comparison post-test (FIG. 1D and FIG. 1E), or unpaired t-test (FIG. 1F) between all groups. *p<0.05 and **p<0.01.

FIGS. 2 A-F. Alum-adjuvanted RSV PreF vaccination elicits a type-2 innate immune response. Mice were challenged, immunized, and re-challenged with RSV as described in FIG. 1. At 4 days post-infection (dpi), BAL was harvested for quantification of eosinophils (FIG. 2A), neutrophils (FIG. 2B), and monocytes (FIG. 2C). Lungs were harvested and homogenized for quantification of ILC2s (FIG. 2D) and ILC2 intracellular cytokine staining of IL-5 (FIG. 2E) and IL-13 (FIG. 2F). Data are represented as mean ± SEM. Statistical significance between vaccination groups was calculated using one-way ANOVA with Tukey’s multiple comparison post-test between all groups. **p<0.01, ***p<0.001, and ****p<0.0001.

FIGS. 3 A-H. Alum-adjuvanted RSV PreF vaccination elicits a type-2 T cell response. Mice were challenged, immunized, and re-challenged with RSV as described in FIG. 1. At 4 or 8 days post infection (dpi), BAL was harvested for quantification of CD4⁺ T cells (FIG. 3A) and T cell intracellular cytokine staining of IFNgamma (FIG. 3B), IL-4 (FIG. 3C), IL-5 (FIG. 3D), and IL-13 (FIG. 3E). CD8⁺ T cells were also quantified from the BAL at 4 and 8 dpi (FIG. 3F) with intracellular cytokine staining of granzyme B (FIG. 3G) and IFN gamma (FIG. 3H). Data are represented as mean ± SEM. Statistical significance between vaccination groups at each time point was calculated using one-way ANOVA with Tukey’s multiple comparison post-test between all groups. *p<0.05, **p<0.01, ***p<0.001, and ****p<0.0001.

FIG. 4. A-F Airway mucus production is elevated in RSV-experienced young mice vaccinated with alum-adjuvanted RSV PreF. Mice were challenged, immunized, and re-challenged with RSV as described in FIG. 1. At 4 or 8 days post infection (dpi), lungs were formalin-filled, paraffin-embedded and sectioned for staining with H&E (FIGS. 4. A, a-f). Lungs were scored, averaged, and inflammatory score graphed. Data is represented as mean ± SEM (n=3) (FIG. 4. B). Inflammatory score trends from 4 to 8 dpi are shown in (FIG. 4. C). PAS staining was also performed at 4 dpi (FIG. 4. D, e-f). To quantify the extent of PAS staining, lungs were scored as described in the methods. Scores were averaged and the total percentage of PAS+ airways were graphed (FIG. 4. E). PAS staining quantification trends from 4 to 8 dpi are shown in (FIG. 4. F). Statistical significance between vaccination groups was calculated in using one-way ANOVA with Tukey’s multiple comparison post-test (FIG. 4. B and FIG. 4. E) or 2-way ANOVA with Sidak’s multiple comparison post-test (FIG. 4. C and FIG. 4. F) between all groups. *p<0.05.

FIG. 5. Inflammation and PAS severity scores. A detailed breakdown of severity scores for each cohort are provided as pie charts, calculated as the number of airways of each severity score (0 - 4) divided by the total number of airways. For inflammatory scores: 0 = 0% field of view involved, 1 = 1-25% involved, 2 = 26-50% involved, 3 = 51-75% involved, and 4 = 76-100% involved. For PAS severity: 0 = 0% of airway is PAS+, 1 = 1-25% PAS+ airway, 2 = 26-50% PAS+ airway, 3 = 51-75% PAS+ airway, and 4 = 75-100% PAS+ airway. The percentages of each severity score were averaged within immunization groups and displayed as pie charts for inflammation and PAS severity at 4 and 8 dpi.

FIG. 6. CD8⁺ T cells produce increased IL-13 in PreF/Advax-SM vaccinated mice. Mice were challenged, immunized, and re-challenged with RSV as described in FIG. 1. At 4 or 8 dpi, BAL was harvested for CD8⁺ T cell intracellular cytokine staining of IL-13. Data are represented as mean ± SEM. Statistical significance between vaccination groups at each time point was calculated using one-way ANOVA with Tukey’s multiple comparison post-test between all groups. *p<0.05, **p<0.01, and ****p<0.0001.

### DETAILED DESCRIPTION

While some of the main embodiments of the present invention are described in the above Summary of the Invention and in the Examples and Claims sections of this patent application, this Detailed Description section provides certain additional description relating to the compositions and methods of the present invention, and is intended to be read in conjunction with all other sections of the present patent application.

### Definitions & Abbreviations

Unless defined otherwise, all technical and scientific terms used herein have the same meaning as commonly understood to one of ordinary skill in the art to which this invention belongs. The following definitions are provided for the full understanding of terms used in this specification.

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Units, prefixes, and symbols are denoted in their Système International de Unites (SI) accepted form. Numeric ranges provided herein are inclusive of the numbers defining the range.

Where a numeric term is preceded by “about” or “approximately,” the term includes the stated number and values ±10% of the stated number.

It should be noted that whenever an embodiment of the present invention refers to a numeric term (or numeric range) with the qualifier “about,” an alternative embodiment having the precise stated numeric value (or numeric range) without the “about” qualifier is also contemplated and also falls within the scope of the present invention. For example, if the patent disclosure refees to an embodiment in which a subject is from about 5 to about 12 years in age, an alternative embodiment in which a subject is from 5 to 12 years in age is also contemplated and also falls within the scope of the present invention. Conversely, it should be noted that whenever an embodiment of the present invention refers to a specific numeric term (or specific numeric range) an alternative embodiment with an “about” qualification is also contemplated and also falls within the scope of the present invention.

Wherever embodiments are described with the language “comprising,” otherwise analogous embodiments described in terms of “consisting of” and/or “consisting essentially of” are included.

Several embodiments of the present invention involve “administration” of specified compositions or agents to subjects. The term “administration” includes any route of introducing or delivering the specified compositions or agents to subjects. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “co-administration,” “administration in combination”, “simultaneous administration” or “administered simultaneously” as used herein, means that the compounds are administered at about the same point in time, overlapping in time, or one following the other. In the latter case, the two compounds are administered at times sufficiently close that the results observed are indistinguishable from those achieved when the compounds are administered at the same point in time. “Systemic administration” refers to introducing or delivering to a subject a specified composition or agent via a route which introduces or delivers the composition or agent to extensive areas of the subject’s body (e.g. greater than 50% of the body), for example through entrance into the circulatory or lymph systems. By contrast, “local administration” refers to introducing or delivering to a subject a specified composition or agents via a route which introduces or delivers the agent to the area or area immediately adjacent to the point of administration and does not introduce the agent systemically in a therapeutically significant amount. For example, locally administered agents are easily detectable in the local vicinity of the point of administration, but are undetectable or detectable at negligible amounts in distal parts of the subject’s body. Administration includes self-administration and the administration by another.

Several of the embodiments of the present invention refer to administration of an “effective amount” of a specified composition or agent. An “effective amount” is an amount sufficient to provide the desired (or stated) effect. For example, an effective amount may be an amount sufficient to prevent RSV infection, or ameliorate RSV infection, or elicit a protective immune response against RSV. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation, such as dose-escalation studies and the like. Also, as used herein, and unless specifically stated otherwise, an “effective amount” of a specified composition or agent can also refer to an amount covering either or both a therapeutically effective amount and a prophylactically effective amount. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

“Identical” or percent “identity,” in the context of two or more nucleic acids or polypeptide sequences, refer to two or more sequences or subsequences that are the same or have a specified percentage of amino acid residues or nucleotides that are the same (e.g., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%,94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the complement of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) amino acid sequence identity is defined as the percentage of amino acids in a candidate sequence that are identical to the amino acids in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=-4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=-4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

“Pharmaceutically acceptable” component can refer to a component that is not biologically or otherwise undesirable, e.g., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

In some embodiments the compositions described herein may comprise a “pharmaceutically acceptable carrier” (sometimes referred to simply as a “carrier”). Suh a “pharmaceutically acceptable carrier” or “carrier” means a substance or excipient that is useful in preparing a composition suitable for administration to a living subject (such as a living human subject) and that is generally safe and non-toxic. The terms “carrier” or “pharmaceutically acceptable carrier” can include, but are not limited to, phosphate buffered saline solutions, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

Several embodiments of the present invention refer to RSV “polypeptides.” The term “polypeptide” is used in its broadest sense to refer to a compound of two or more subunit amino acids, amino acid analogs, or peptidomimetics. Typically, the subunits may be linked by peptide bonds. As used herein the term “amino acid” refers to either natural and/or unnatural or synthetic amino acids, including glycine and both the D or L optical isomers, and amino acid analogs and peptidomimetics.

The term “vaccine” as used herein refers to a composition comprising an RSV F polypeptide as described herein, which is useful to provide one or more of the outcomes described herein - e.g. to prevent RSV infection, ameliorate RSV infection, elicit a protective immune response against RSV and the like. Typically such vaccine compositions will comprise an RSV F polypeptide as described herein and a pharmaceutically acceptable carrier. In some embodiments such vaccine compositins will also comprise an adjuvant.

### Compositions

It is understood that the compositions described herein can be used in combination with the various other compositions and agents that that they can also be used in carrying out the various methods disclosed herein.

Disclosed herein are various compositions comprising a RSV F polypeptide stabilized in a prefusion conformation (which may be referred to herein as “preF”). The RSV F or “fusion” protein is an envelope polypeptide of RSV viruses. A homotrimer of RSV F proteins mediates fusion of viral and cellular membranes during RSV infection. The RSV F polypeptide described herein can be derived from any RSV subtype (e.g., subtypes A or B) or from any isolate which is a clinical, laboratory/engineered, non-virulent, or non-infectious isolate. A wild-type RSV F polypeptide from a RSV subtype A virus can comprise an amino acid sequence of SEQ ID NO:1, and wild-type F polypeptide from a RSV subtype B virus can comprise an amino acid sequence of SEQ ID NO:2. One of skill in the art will appreciate that wild-type sequences are merely one of many possible sequences, and that numerous other wild-type or engineered RSV F polypeptide having amino acid sequence different from those of SEQ ID NO:1 and SEQ ID NO:2 can be used, including, but not limited to, those specific mutant RSV F polypeptide molecules and sequences described herein.

The RSV F protein exists in at least two conformers: a prefusion and a postfusion conformation. Upon binding of the virus to a host cell, the F-protein undergoes a conformational change from a prefusion to a postfusion conformation. Prefusion F-protein is the primary determinant of neutralizing activity against RSV in human sera, but soluble prefusion F-protein is highly unstable and readily converts to a postfusion conformation. Accordingly, included herein are RSV F polypeptides which are stabilized in a pre-fusion conformation. The three-dimensional structure of an example RSV F protein in a prefusion conformation is disclosed in U.S. Pat. Application Publication US20160046675.

As used herein, the terms “RSV F polypeptide stabilized in a prefusion conformation” and “preF” include synthetic and engineered RSV F polypeptides, including, but not limited to, those described in U.S. Pat. Application Publications US20140271699, US20150030622, US20160046675, US2017/0182151, U.S. Pat. No. 9,738,689, and International Patent Application Publications WO2015/013551 and WO2019/032480, the content of each of which - including their sequence listings, is hereby incorporated by reference in their entireties for those jurisdictions that permit incorporation by reference. In some embodiments, the RSV F polypeptide stabilized in a prefusion conformation is in a soluble form.

The term “stabilized” as it refers to a prefusion conformation of a RSV F polypeptide, refers to an increased stability of a prefusion conformation resulting from a modification, as compared to the stability of the prefusion conformation without the modification. Absolute stability is expressly not required; rather the modification introduces an increased degree of stability in a prefusion conformation. Stability, and relative stability, may be measured in various ways, for example by measuring the half-life of the RSV pre-fusion conformation. The increased instability may be to any degree that is useful or significant for the intended application. For example, stability may be increased by about 10%, 25%, 50%, 100%, 200% (i.e. 2-fold), 300% (i.e. 3-fold), 400% (i.e. 4-fold), 500% (i.e. 5-fold), 1000% (i.e. 10-fold), or more.

An RSV F polypeptide stabilized in a prefusion conformation can be described by its physical and/or functional attributes. In some embodiments, an RSV F polypeptide stabilized in a prefusion conformation contains a unique antigenic site referred to as “antigenic site Ø.” The antigenic site Ø is located at the membrane-distal apex of the F protein when in a prefusion conformation, but elements of antigenic site Ø reposition in a postfusion conformation such that antibodies (e.g., D25 and AM22) cannot specifically bind the site. The antigenic site Ø can comprise amino acids 62-69 and 196-209 of a wild-type RSV F protein sequence (e.g., SEQ ID NO:1 or SEQ ID NO:2) or can comprise any antigenic site Ø sequence disclosed in US20150030622, US20160046675 or WO2019/032480.

An RSV F polypeptide stabilized in a prefusion conformation can be specifically bound by an antibody that is specific for the prefusion conformation of the RSV F protein and does not bind a postfusion conformation, such as an antibody that specifically binds to an epitope within antigenic site Ø, for example, a D25, AM22, or 5C4 antibody. Methods to determine whether a F protein contains a prefusion epitope (e.g., a D25 epitope or AM22 epitope) are disclosed in U.S. Pat. Application Publications US20100068217, incorporated by reference herein in its entirety, and in US20160046675. Heavy and light chain amino acid sequences of a D25 monoclonal antibody are disclosed in U.S. Pat. Application Publication

US20100239593, incorporated by reference herein in its entirety, and further disclosed in Kwakkenbos et al., Nat. Med., 16:123-128 (2009). Heavy and light chain amino acid sequences of an AM22 monoclonal antibody are disclosed in U.S. Pat. Application Publication US20120070446, incorporated by reference herein in its entirety, and the specificity of AM22 for prefusion F protein is disclosed in U.S. Pat. Application Publication US20160046675. Heavy and light chain amino acid sequences of a 5C4 monoclonal antibody are disclosed in U.S. Pat. Application Publication US20160046675 and in McLellan et al., Science, 340(6136): 1113-7 (2013).

Alternatively, an RSV F polypeptide stabilized in a prefusion conformation can be specifically bound by an antibody specific for the prefusion conformation of the RSV F protein but which does not bind antigenic site Ø, for example a MPE8 antibody. Heavy and light chain amino acid sequences of a MPE8 monoclonal antibody are disclosed in U.S. Pat. Application Publication US20160046675 and further discussed in Corti et al., Nature, 501(7467):439-443 (2013).

Conversely, a postfusion conformation differs in three-dimensional folding of the RSV F polypeptide and is described in U.S. Pat. Application Publication US20160046675, and further described at the atomic level in McLellan et al., J. Virol., 85:7788 (2011); Swanson et al., Proc. Natl. Acad. Sci., 108:9619 (2011); and in which structural coordinates are deposited and available at Protein Data Bank Accession No. 3RRR. A postfusion conformation does not include a D25 epitope, a AM22 epitope, or the same antigenic site Ø spatial arrangement as the prefusion conformation, and thus is not specifically bound by D25 or AM22 antibodies. A RSV F protein stabilized in a prefusion conformation can also be identified in some embodiments by the absence of binding by an antibody which specifically binds a postfusion conformation but does not bind a prefusion conformation. For example, an antibody which specifically binds the six-helix bundle present only in a postfusion conformation and not in a prefusion conformation does not specifically bind a RSV F protein stabilized in a prefusion conformation. An example of a postfusion-specific antibody is described in Magro et al., Proc. Natl′l. Acad. Sci., 109:3089-94 (2012).

In some embodiments, the RSV F polypeptide stabilized in a prefusion conformation comprises a modification capable of forming one or more non-natural disulfide bonds, for example, the addition of or substitution by one or more cysteine residues. RSV F polypeptides containing one or more modifications that create non-natural disulfide bonds in the RSV F polypeptide or between RSV F polypeptides are referred to herein with nomenclature that includes the letters “DS.” A non-natural disulfide bond is one that does not occur in a native RSV F protein, and is introduced by protein engineering (e.g., by including one or more substituted cysteine residues that contribute to the formation of the non-natural disulfide bond). Examples of non-natural disulfide bond-forming modifications are described for RSV F polypeptide in U.S. Pat. Application Publications US20150030622 and US20160046675. In some embodiments, the RSV F polypeptide comprises S155C and S290C amino acid substitutions which can form a disulfide bond. The S155C/S290C-substituted RSV F polypeptide is referred to herein as “DS,” as further described in U.S. Pat. Application Publications US20150030622 and US20160046675.

Accordingly, included herein are RSV F polypeptides stabilized in a prefusion conformation comprising one or more disulfide bonds which can stabilize the F polypeptide in a prefusion conformation. In some embodiments, the RSV F polypeptide can be an aqueous-soluble polypeptide comprising S155C/S290C-substitutions. The RSV F polypeptide can comprise the amino acid sequence of SEQ ID NO: 3, or a polypeptide sequence having at or greater than about 80%, at or greater than about 85%, at or greater than about 90%, at or greater than about 95%, or at or greater than about 98% homology with SEQ ID NO: 3. In some embodiments, the RSV F polypeptide can be a full-length polypeptide comprising S155C/S290C-substitutions. In some embodiments, the RSV F polypeptide comprises the amino acid sequence of SEQ ID NO: 4, or a polypeptide sequence having at or greater than about 80%, at or greater than about 85%, at or greater than about 90%, at or greater than about 95%, or at or greater than about 98% homology with SEQ ID NO: 4.

In some embodiments, the RSV F polypeptide stabilized in a prefusion conformation comprises a modification capable of forming one or more dityrosine bonds, for example, by the addition of or substitution by one or more tyrosine residues. RSV F polypeptides containing one or more modifications that create dityrosine bonds in the RSV F polypeptide or between RSV F polypeptides are referred to herein with nomenclature that includes the letters “DT.” Numerous dityrosine bond-forming modifications are described for RSV F polypeptide in U.S. Pat. Application Publication US20150030622 and International Patent Application Publication WO/2019/032480 - each being incorporated by reference herein. In some embodiments, the RSV F polypeptide comprises a to-tyrosine (i.e. to “Y”) mutation at one or more of the following amino acid position (numbered by alignment to SEQ ID NO. 1): 77Y, 88Y, 97Y, 147Y, 150Y, 155Y, 159Y, 183Y, 185Y, 187Y, 220Y, 222Y, 223Y, 226Y, 255Y, 427Y, 428Y and 469Y amino acid substitutions which can form a dityrosine bond. The dityrosine bond can be between an existing wild-type tyrosine residue (e.g., Y33, Y198, and Y286) and a tyrosine-substituted or inserted residue, or between two tyrosine-substituted or inserted residues. In some embodiments, the RSV F-polypeptide comprises one or more of 77Y, 185Y, 222Y, 226Y, 427Y, 428Y and 469Y amino acid substitutions - any of which can provide a site for dityrosine bond formation. In some embodiments, the RSV F-polypeptide comprises one or more of 185Y, 226Y and 428Y amino acid substitutions - any of which can provide a site for dityrosine bond formation.

Accordingly, included herein are RSV F polypeptides comprising one or more dityrosine bonds which can stabilize the F polypeptide in a prefusion conformation. In some embodiments, the RSV F polypeptide can be an aqueous-soluble polypeptide comprising K77Y/E222Y substitutions. In some embodiments, the RSV F polypeptide comprises the amino acid sequence of SEQ ID NO: 5, or a polypeptide sequence having at or greater than about 80%, at or greater than about 85%, at or greater than about 90%, at or greater than about 95%, or at or greater than about 98% homology with SEQ ID NO: 5. In some embodiments, the RSV F polypeptide can be a full-length polypeptide comprising K77Y/E222Y substitutions.

In some or further embodiments, the RSV F polypeptide stabilized in a prefusion conformation comprises one or more amino acid substitutions which partially or completely fill a cavity within the F polypeptide or between F polypeptides. Polypeptides containing one or more such cavity mutations are referred to herein with nomenclature that includes the letters “CAV.” The cavity can be between protomers of the RSV F protein, and can be a cavity present in a prefusion conformation which collapses (e.g., has reduced volume) after transition to a postfusion conformation. In some embodiments, the RSV F-polypeptide comprises one or more of S190F and V207L amino acid substitutions which can stabilize the F polypeptide in a prefusion conformation. A S190F/V207L-substituted RSV F polypeptide is referred to herein as “Cav1” and is further described in U.S. Pat. Application Publications US20150030622 and US20160046675. In some embodiments, the RSV F polypeptide can be an aqueous-soluble polypeptide comprising S190F/V207L-substitutions. In some embodiments, the RSV F polypeptide comprises the amino acid sequence of SEQ ID NO: 6, or a polypeptide sequence having at or greater than about 80%, at or greater than about 85%, at or greater than about 90%, at or greater than about 95%, or at or greater than about 98% homology with SEQ ID NO: 6. In some embodiments, the RSV F polypeptide can be a full-length polypeptide comprising S190F/V207L-substitutions. In some embodiments, the RSV F polypeptide comprises the amino acid sequence of SEQ ID NO: 7, or a polypeptide sequence having at or greater than about 80%, at or greater than about 85%, at or greater than about 90%, at or greater than about 95%, or at or greater than about 98% homology with SEQ ID NO: 7.

An RSV F polypeptide can contain one or more combinations of modifications which stabilize the polypeptide in the prefusion conformation. For example, included herein are RSV F polypeptides containing two or more of DS, DT and CAV mutations. In some embodiments, the RSV F polypeptide comprises S190F, V207L, S155C, and S290C amino acid substitutions and is referred to herein as “DS-Cav1,” as further described in U.S. Pat. Application Publications US20150030622 and US20160046675. In some embodiments, the RSV F polypeptide can be an aqueous-soluble polypeptide comprising S190F/V207L/S155C/S290C amino acid substitutions. In some embodiments, the RSV F polypeptide comprises the amino acid sequence of SEQ ID NO: 8, or a polypeptide sequence having at or greater than about 80%, at or greater than about 85%, at or greater than about 90%, at or greater than about 95%, or at or greater than about 98% homology with SEQ ID NO: 8. In some embodiments, the RSV F polypeptide can be a full-length polypeptide comprising S190F/V207L/S155C/S290C amino acid substitutions. In some embodiments, the RSV F polypeptide comprises the amino acid sequence of SEQ ID NO: 9, or a polypeptide sequence having at or greater than about 80%, at or greater than about 85%, at or greater than about 90%, at or greater than about 95%, or at or greater than about 98% homology with SEQ ID NO: 9.

In some embodiments, the RSV F polypeptide stabilized in a prefusion conformation further comprises a trimerization domain as described in U.S. Pat. Application Publications US20150030622 and US20160046675, which domain allows for trimerization of the RSV F polypeptide. The trimerization domain can be referred to as a Foldon domain. Accordingly, in some embodiments, the RSV F polypeptide is a homotrimer. The trimerization domain can comprise any trimerization domain polypeptide sequence, and can be encoded by any trimerization domain polynucleotide sequence, disclosed in U.S. Pat. Application Publications US20150030622 and US20160046675.

Different monomers of an RSV F polypeptide stabilized in a prefusion conformation can, in some embodiments, be trimerized by inclusion of a trimerization domain, resulting in a heterotrimer (e.g., a heterotrimer of one monomer each of DS, DT, and Cav1). In such embodiments, the heterotrimer is stabilized in a prefusion conformation by one or more modifications. In some or further embodiments, a vaccine composition for vaccination against RSV can comprise a mixture of two or more RSV F polypeptides stabilized in a prefusion conformation and an inulin adjuvant (e.g., a mixture of “DSCav1” F polypeptides and “DTCav1” F polypeptides).

The compositions can comprise an RSV F polypeptide stabilized in a prefusion conformation in various amounts. The composition can comprise the RSV F polypeptide in an amount ranging from about 1 ng/mL to about 1 g/mL. In some embodiments, the composition comprises RSV F polypeptide in an amount ranging from about 10 ng/mL to about 100 mg/mL, from about 100 ng/mL to about 10 mg/mL, from about 100 ng/mL to about 1 mg/mL, from about 1 µg/mL to about 1 mg/mL, or from about 10 µg/mL to about 1 mg/mL.

Suitable carriers or excipients that can be used include, but are not limited to, salts, diluents, (e.g., Tris-HCl, acetate, phosphate), preservatives (e.g., Thimerosal, benzyl alcohol, parabens), binders, fillers, solubilizers, disintegrants, sorbents, solvents, pH modifying agents, antioxidants, antinfective agents, suspending agents, wetting agents, viscosity modifiers, tonicity agents, stabilizing agents, and other components and combinations thereof. Suitable pharmaceutically acceptable excipients are preferably selected from materials which are generally recognized as safe (GRAS), and may be administered to an individual without causing undesirable biological side effects or unwanted interactions. Suitable excipients and their formulations are described in Remington’s Pharmaceutical Sciences, 16th ed. 1980, Mack Publishing Co. In addition, such compositions can be complexed with polyethylene glycol (PEG), metal ions, or incorporated into polymeric compounds such as polyacetic acid, polyglycolic acid, hydrogels, etc., or incorporated into liposomes, microemulsions, micelles, unilamellar or multilamellar vesicles, erythrocyte ghosts or spheroblasts. Suitable dosage forms for administration, e.g., parenteral administration, include solutions, suspensions, and emulsions. Typically, the components of the vaccine formulation are dissolved or suspended in a suitable solvent such as, for example, water, Ringer’s solution, phosphate buffered saline (PBS), or isotonic sodium chloride. The formulation may also be a sterile solution, suspension, or emulsion in a nontoxic, parenterally acceptable diluent or solvent such as 1,3-butanediol. In some cases, formulations can include one or more tonicity agents to adjust the isotonic range of the formulation. Suitable tonicity agents are well known in the art and include glycerin, mannitol, sorbitol, sodium chloride, and other electrolytes. In some cases, the formulations can be buffered with an effective amount of buffer necessary to maintain a pH suitable for parenteral administration. Suitable buffers are well known by those skilled in the art and some examples of useful buffers are acetate, borate, carbonate, citrate, and phosphate buffers. In some embodiments, the formulation can be distributed or packaged in a liquid form, or alternatively, as a solid, obtained, for example by lyophilization of a suitable liquid formulation, which can be reconstituted with an appropriate carrier or diluent prior to administration.

In many embodiments the methods and compositions described herein involve Th-balanced adjuvants. The term “adjuvant” as used herein, refers to a substance that, when administered to a subject, increases the subject’s immune response to a vaccine immunogen - i.e. administering both a vaccine immunogen and an adjuvant to a subject results in a greater immune response in the subject than is achieved if the vaccine immunogen is administered without an adjuvant. In some embodiments of the present invention adjuvants are included in the same composition as the vaccine immunogen (i.e. the same composition as the RSV F polypeptide). In some embodiments of the present invention the adjuvants are provided in a separate composition - i.e. not in the same composition as the RSV F polypeptide. In such embodiments a composition comprising an RSV F polypeptide and a composition comprising an adjuvant may be co-administered to a subject (i.e. at approximately the same time) or they may be administered to a subject at different times (for example separated by minutes, hours, or days).

The term “Th-balanced adjuvant” refers to an adjuvant that induces Th1-type CD8 responses, high levels of inflammatory IFNgamma, and Th2-mediated increases in antibody production simultaneously in a subject.

Any Th-balanced adjuvant known in the art can be used in the methods and compositions of the present invention. Examples of Th-balanced adjuvants that can be used include, but are not limited to, CpG oligonucleotides, MPL, Freunds Complete Adjuvant, saponin, dQS21, poly(I:C), poly(IC:LC), oil-in-water emulsion adjuvants and Advax-SM (Advax-SM is an inulin adjuvant comprising CpG oligonucleotides). Advax is further described in U.S. Patent Application Publication US 20170239349, WIPO Patent Application Publication WO2012175518, and Australian Patent Application Publication AU2017203501, each of which are incorporated herein in their entireties. Additional description of Th-balanced adjuvants and the differences between Th-balanced and Th2-skewed adjuvants is provided in: Sastry et al. “Adjuvants and the vaccine response to the DS-Cav1-stabilized fusion glycoprotein of respiratory syncytial virus.” PLoS One. 2017;12:e0186854; Culley et al., “Age at first viral infection determines the pattern of T cell-mediated disease during reinfection in adulthood.” J Exp Med. 2002;196:1381-6; Cerwenka et al., “Migration kinetics and final destination of type 1 and type 2 CD8 effector cells predict protection against pulmonary virus infection.” J Exp Med. 1999;189:423-34; and Eichinger et al., “Prefusion RSV F Immunization Elicits Th2-Mediated Lung Pathology in Mice When Formulated With a Th2 (but Not a Th1/Th2-Balanced) Adjuvant Despite Complete Viral Protection; Frontiers in Immunology; 2020; 11, 1673 - the contents of each of which are hereby incorporated by reference herein.

The compositions can comprise an adjuvant in various amounts. The composition can comprise an adjuvant in an amount ranging from about 1 ng/mL to about 1 g/mL. In some embodiments, the composition comprises an adjuvant in an amount ranging from about 10 ng/mL to about 100 mg/mL, from about 100 ng/mL to about 10 mg/mL, from about 100 ng/mL to about 1 mg/mL, from about 1 µg/mL to about 1 mg/mL, or from about 10 µg/mL to about 1 mg/mL.

### Methods

The present invention provides various methods for preventing or ameliorating respiratory syncytial virus (RSV) infection or eliciting a protective immune response against RSV infection in juvenile subjects, such methods comprising administering to juvenile subjects an effective amount of: (a) an RSV F polypeptide stabilized in a prefusion conformation, and (b) a Th-balanced adjuvant, thereby preventing, ameliorating, or eliciting a protective immune response against respiratory syncytial virus (RSV) infection in such subjects. In the case of ameliorating respiratory syncytial virus (RSV) infection, the amelioration may, for example, constitute any detectable or measurable or clinically meaningful decrease in degree of infection, duration of infection, severity of infection, symptoms of infection, viral load, or any other clinically relevant measure of RSV infection. In some situations, prevention or amelioration of RSV infection or elicitation of a protective immune response against RSV infection, may be ascertained in comparison to a control - e.g., a control subject or a control group of subjects.

The RSV F polypeptide can be any RSV F polypeptide stabilized in a prefusion conformation known in the art or disclosed herein.

Similarly, the Th-balanced adjuvant can be any Th-balanced adjuvant known in the art or disclosed herein.

The RSV infection may be caused by any RSV virus capable of causing infection (e.g., capable of infecting a subject, thereby resulting in a clinical diagnosis of RSV infection). In some embodiments, the RSV is a human RSV. In some embodiments, the RSV is a subtype A virus (e.g., GA1, GA2, GA3, GA4, GA5, GA6, GA7, SAA1, NA1, NA2, NA3, NA4, ON1, or any combination thereof). In some the RSV is a subtype B virus (e.g., GB1, GB2, GB3, GB4, SAB1, SAB2, SAB3, SAB4, URU1, URU2, BA1, BA2, BA3, BA4, BA5, BA6, BA7, BA8, BA9, BA10, BA-C, THB, or any combination thereof).

The subject can be any mammalian subject, for example a human, dog, cow, horse, mouse, rabbit, etc. In some embodiments, the subject is a primate. In some embodiments, the subject is a human. The subject can be a male or female.

The RSV F polypeptide stabilized in a prefusion conformation and the Th-balanced adjuvant can be administered to the subject together or separately. In some embodiments, the RSV F polypeptide and the adjuvant are administered within a four-week period, within a three-week period, within a two-week period, or within a one-week period of each other. In some embodiments, the RSV F polypeptide and the adjuvant are administered within a six-day period, within a five-day period, within a four-day period, within a three-day period, or within a two-day period of each other. In some embodiments, the RSV F polypeptide and the adjuvant are administered within a 24-hour period, within a 12-hour period, within a 6-hour period, within a 3-hour period, or within a 1-hour period of each other. In some embodiments, the RSV F polypeptide and the adjuvant are administered concurrently, for example, in the same composition. In some embodiments, the RSV F polypeptide and the adjuvant are administered together in the same composition - e.g. a composition comprising the RSV F polypeptide, the adjuvant and a pharmaceutically acceptable carrier.

The methods can include more than one administration of the RSV F polypeptide, the adjuvant, or both, for example as a part of a so-called prime-boost vaccination protocol. In some embodiments there may be at least one, at least two, at least three, at least four, at least five, or more administrations of the RSV F polypeptide and/or adjuvant.

In some embodiments, a subsequent administration is provided at least one week after a prior administration. In some embodiments, a subsequent administration is provided at least two weeks, at least three weeks, or at least four weeks after a prior administration. In some embodiments, a subsequent administration is provided at least one month, at least two months, at least three months, at least six months, or at least twelve months after a prior administration.

The amount of the disclosed compositions administered to a subject will vary from subject to subject, depending on the nature of the disclosed compositions and/or vaccine formulations, the species, gender, age, weight and general condition of the subject, the mode of administration, and the like. Effective dosages and schedules for administering the compositions may be determined empirically, and making such determinations is within the skill in the art. The dosage ranges for the administration of the disclosed compositions and vaccine formulations are those large enough to produce the desired effect (e.g., to reduce RSV infection). The dosage should not be so large as to cause adverse side effects, such as unwanted cross-reactions, anaphylactic reactions, and the like. The dosage can be adjusted by the individual physician in the event of any counterindications. Generally, the disclosed compositions and/or vaccine formulations are administered to the subject at a dosage of active component(s) ranging from 0.1 µg/kg body weight to 100 g/kg body weight. In some embodiments, the disclosed compositions and/or vaccine formulations are administered to the subject at a dosage of active component(s) ranging from 1 µg/kg to 10 g/kg, from 10 µg/kg to 1 g/kg, from 10 µg/kg to 500 mg/kg, from 10 µg/kg to 100 mg/kg, from 10 µg/kg to 10 mg/kg, from 10 µg/kg to 1 mg/kg, from 10 µg/kg to 500 µg/kg, or from 10 µg/kg to 100 µg/kg body weight. Dosages above or below the range cited above may be administered to the individual patient if desired.

In some embodiments, the method reduces RSV infection in a subject (or group of subjects) as compared to a control subject (or control group of subjects). In some embodiments, the method reduces RSV infection in a subject (or group of subjects) by at least 25%, at least 50%, or at least 75% as compared to a control subject (or control group of subjects). In some embodiments, the method reduces RSV infection in a subject (or group of subjects) by at least one-fold, at least two-fold, at least three-fold, at least four-fold, or at least five-fold as compared to a control subject (or control group of subjects). In some embodiments, the method reduces RSV infection in a subject (or group of subjects) by at least one log, at least two logs, at least three logs, at least four logs, at least five logs, or at least six logs as compared to a control subject (or control group of subjects). In some embodiments, the method reduces RSV infection in a subject to below a detectable level.

The presence and/or extent/degree/amount of RSV infection can be determined in a biological sample from a subject (or group of subjects). The biological sample may be blood, plasma, serum, nasal swab, mucosal mouth or airway swab, sputum, tissue biopsy, or other suitable biological samples comprising RSV. The amount of RSV can be determined in the biological sample by, for instance, direct measurement of RSV particles (e.g., in a plaque assay) or portions thereof (antigens in e.g., a RSV-specific ELISA). In some embodiments, the amount of RSV infection can be determined by indirect measurements in a biological sample, such as detection of RSV-specific immunoglobulins or measurements of leukocyte counts. Alternatively, the amount of RSV infection can be determined by methods which do not require obtaining a biological sample (e.g., chest X-ray, skin pulse oximetry, general clinician observation, etc.).

The amount of RSV infection can be compared to a control. The control can be a biological sample from, for example, a cell line, a tissue stock, etc., or alternatively can be a subject (e.g., an unvaccinated subject). The control can alternatively be a subject, or a biological sample therefrom, which is vaccinated using a different vaccine or different vaccination method. A control should also be infected/challenged with a similar titer of RSV. In some embodiments, a control for comparing the amount of RSV infection can be a subject, or a biological sample therefrom, administered with a vaccine composition comprising a RSV F polypeptide and a non-inulin adjuvant. Alternatively, a control can be a collection of values used as a standard applied to one or more subjects (e.g., a general number or average that is known and not identified in the method using a sample).

One advantage of the disclosed methods is that the methods can increase the safety of anti-RSV vaccination compared to methods using other presently known vaccines or vaccine candidates. In some embodiments, the method decreases eosinophilia in the subject as compared to a control. In some embodiments, essentially no clinical eosinophilia results in the subject after performing the methods. In some embodiments, the method decreases vaccine-enhanced respiratory disease (VERD; also known as enhanced respiratory disease (ERD) or vaccine enhanced disease (VED)) in the subject as compared to a control. In some embodiments, essentially no clinical VERD results in the subject after performing the methods. A control to which measures of safety can be compared can include a vaccinated subject using a different vaccine or different vaccination method, or a biological sample therefrom. In some embodiments, a control for comparing safety can be a subject, or a biological sample therefrom, administered with vaccine composition comprising an RSV F polypeptide and a non-Th-balanced adjuvant. A control for comparing safety can be, but need not be, infected/challenged with RSV. Alternatively, a control for comparing safety can be a collection of values used as a standard applied to one or more subjects (e.g., a general number or average that is known and not identified in the method using a sample).

In some embodiments, the methods result in desirable cellular and immunological responses. In some embodiments, the subject can have reduced Fc receptor expression on natural killer cells. In some embodiments, the subject can have reduced Scavenger Receptor A (SR-A) expression and/or increased major histocompatibility complex class II (MHCII) expression on resting alveolar macrophages. In some embodiments, the subject can have reduced eosinophil levels. In some embodiments, the subject can have increased CD8+ T cell levels, increased CD4+ T cell levels, or any combination thereof. In some embodiments, the subject can have reduced levels of interleukin 4 (IL-4), interleukin 5 (IL-5), interleukin 13 (IL-13), or any combination thereof. In some embodiments, the subject can have increased interferon gamma levels. In some embodiments, the subject can have increased anti-RSV F-polypeptide IgG antibody levels. In some embodiments, the subject can have an increased ratio of Th1:Th2 cell responses (e.g., increased ratio of Th1:Th2 cell levels). In some embodiments, the administration of the adjuvant increases the ratio of Th1:Th2 cell responses in the subject as compared to a control. In some embodiments, the desirable cellular and immunological responses are measurable at least in bronchioalveolar lavage fluid (BALF).

The invention is further described by the following non-limiting “Example” and the Figures referred to therein. The numbers in parentheses in this Example section indicate the numbered references in the Reference List section of this disclosure.

### EXAMPLE

### Formulation of the Prefusion RSV F Protein With a Th1/Th2-Balanced Adjuvant Provides Complete Protection Without Th2-Skewed Immunity in RSV-Experienced Young Mice

Respiratory syncytial virus (RSV) is a leading cause of lower respiratory tract infections among infants with most infections occurring in the first year of life. Multiple RSV exposures are required for children to mount adult-like immune responses. Although adult RSV immunity is associated with less severe disease, the protection induced through natural infection is short-lived. Therefore, vaccination of RSV-experienced young children may accelerate immunity and provide long-term protection from RSV reinfection. However, the extent to which different Th-biased vaccine regimens influence pre-existing humoral and cellular immunity in RSV-experienced young children is unknown. To address this question, infant BALB/c mice were RSV-infected and subsequently immunized with the prefusion RSV F (PreF) antigen formulated with either a Th2-skewing (Alum) or Th1/Th2-balanced (Advax-SM) adjuvant. These studies show that both adjuvants boosted neutralizing antibody and protected from RSV reinfection, but Advax-SM adjuvant prevented the Th2-skewed immunity observed in RSV-experienced young mice immunized with PreF/Alum.

In the first year of life, approximately 70% of infants are infected with RSV and by two years of age, 50% of children have been infected multiple times [1]. Humoral immunity is largely dependent on neutralizing antibody directed against RSV F protein and 3-6 seasons of RSV exposure are required for children’s serum neutralizing antibody titers to reach levels comparable to those seen in adulthood [2]. Furthermore, infant RSV memory T cell responses are insufficient to prevent reinfection [3] and IFNgamma-producing T cells are reduced and delayed compared to adults [2]. Thus, a RSV vaccine that accelerates humoral and cellular immunity in RSV-experienced children may confer protection from RSV reinfections. However, the ability of such a vaccine to safely and effectively alter pre-existing infant RSV immunity has not yet been evaluated.

To determine the extent to which RSV F protein subunit immunization affects pre-existing humoral and cellular immunity as well as safety and efficacy, infant BALB/c mice were RSV infected and immunized 3 weeks later with the prefusion conformation of RSV F protein (PreF) formulated with Alum (Th2-polarizing) or Advax-SM (Th1/Th2-balanced) adjuvants. Neutralizing and PreF-specific antibody titers were equivalent among both groups of immunized mice with complete viral protection following RSV challenge. PreF/Alum immunization elicited robust Th2 immunity and increased mucus production, whereas PreF/Advax-SM immunization increased cytolytic CD8⁺ T cells. Together, these data demonstrate that despite pre-existing immunity generated during infant RSV infection, adjuvants with different Th profiles boost antibody responses and produce discrete cellular immunity when used in PreF immunization of RSV-experienced young mice.

### 2. Materials and Methods

### 2.1 Mice, Vaccine Administration, and Viral Quantification

Infant mice born to Balb/cJ dams (The Jackson Laboratory, Bar Harbor, ME) were infected with 5x10⁵ pfu/gm RSV L19 at post-natal day 5-6, as previously described [4]. Three weeks later, mice were primed via intramuscular (i.m.) injection (0.37″ needle) with 50 µl of vehicle (PBS), RSV PreF (DS-Cav1) (10 µg/mouse) formulated with Advax-SM™ (Vaxine Pty Ltd, Bedford Park, Australia) or alum and boosted with their respective vaccine formulation 3 weeks later. At 1-week post-boost, mice were intranasally (i.n.) challenged with 5x10⁵ pfu/gm RSV L19 and culled at 4- or 8-days post-infection (dpi). RSV L19 was propagated and viral titers quantified as previously described [5].

### 2.2 Cell Preparation, Stimulation, and Flow Cytometry

Bronchoalveolar lavage (BAL) and lower right lung lobes were collected, processed, and enumerated, as previously described [6]. Cells were stimulated and processed for flow cytometry. Samples were run on a BD LSRFortessa. Data was analyzed using FlowJo V10 software (FLOWJO, LLC, OR).

### 2.3 Histology

Left lungs were gravity-filled with 10% formalin at 4- and 8 dpi, as previously described [7]. Lungs were processed and stained with hematoxylin and eosin or Periodic Acid-Schiff (PAS). Lung inflammation and mucus hypersecretion were quantified, as previously described [4, 8].

### 2.4 Neutralizing and RSV-specific IgG Subtype

Serum was collected via submandibular bleed 2-3 days prior to secondary RSV challenge and separated using Gel-Z Serum Separator Tubes (Sarstedt, Germany). Serum was stored at -80° C. until heat inactivation (56° C. for 30 minutes). Neutralizing antibody titers were determined using a Renilla Luciferase RSV reporter assay; RSV PreF-specific IgG subtypes were determined via ELISA.

### 2.6 Statistical Analysis

Statistical analysis was performed using GraphPad Prism 8 software (GraphPad Software, La Jolla, CA). Results are displayed as the mean ± SEM and p values <0.05 were considered significant.

### 3. Results

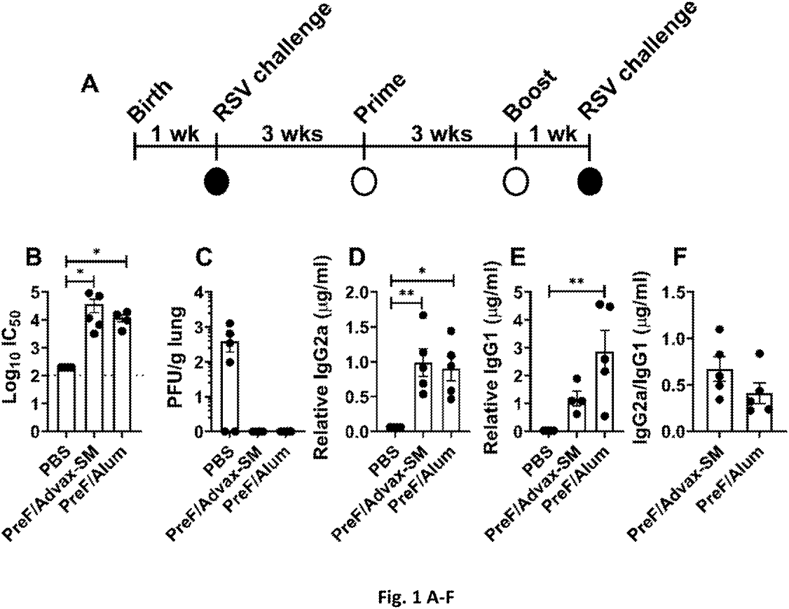

3.1. RSV PreF-immunization of RSV-experienced young mice increases neutralizing antibody titers. To determine the extent to which antibody responses were increased, RSV-experienced young mice were immunized with RSV-PreF adjuvanted with alum (Th2-skewing) or Advax-SM (Th1/Th2-balanced) and serum was collected immediately prior to secondary challenge (FIG. 1A). PreF/Advax-SM and PreF/Alum immunization increased RSV neutralizing antibodies relative to the PBS group and had undetectable virus in the lungs at 4 days post infection (dpi) (FIGS. 1B-C). PreF/Advax-SM and PreF/Alum groups had elevated levels of IgG2a compared to PBS mice, whereas only PreF/Alum-vaccinated animals had increased IgG1 (FIGS. 1D-E). Both PreF/Advax-SM and PreF/Alum groups had ratios < 1 (FIG. 1F), suggesting a Th2-skewed response.

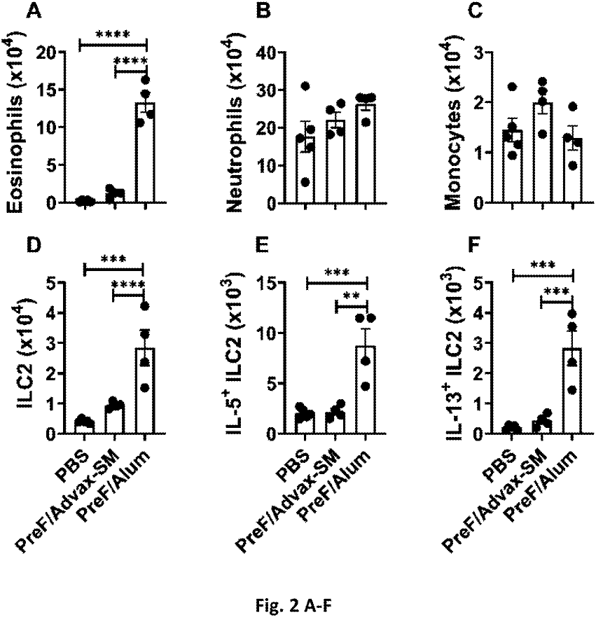

3.2. PreF/Alum elicits Th2-associated innate immunity in RSV-experienced young mice. To elucidate differential cellular responses in PreF/Advax-SM- and PreF/Alum-immunized mice, innate immune cells were quantified in bronchoalveolar lavage (BAL) and lung at 4 dpi. In the BAL, eosinophils were dramatically increased in PreF/Alum-immunized animals, whereas neutrophil and monocyte populations did not differ significantly across groups (FIGS. 2A-C). In lung, type 2 innate lymphoid cells (ILC2) and ILC2s producing IL-5 and IL-13 increased in PreF/Alum animals as compared to PBS and PreF/Advax-SM (FIGS. 2D-F). Collectively, increased eosinophils and activated ILC2s suggest that PreF/Alum, but not PreF/Advax-SM immunization of RSV-experienced young mice induced a Th2-associated innate cellular profile.

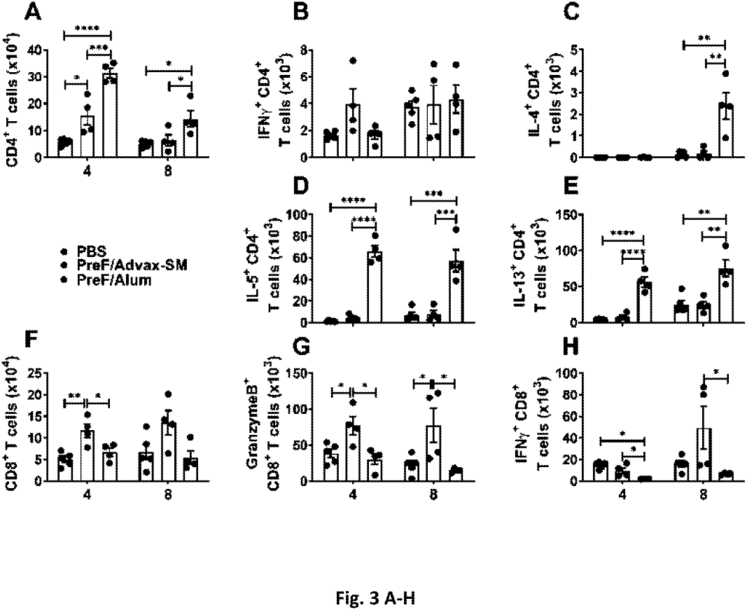

3.3. PreF/Alum generates a CD4⁺ Th2 response, while PreF/Advax-SM promotes cytotoxic CD8⁺ T cells. To determine if T cell responses correlate with the Th2-associated innate immunity observed in PreF/Alum immunized mice, T-helper subtypes were analyzed from the BAL. More CD4⁺ T cells were recovered from PreF/Advax-SM and PreF/Alum-vaccinated mice at 4 dpi and remained elevated in PreF/Alum mice at 8 dpi compared to PBS and PreF/Advax-SM groups (FIG. 3A). PreF/Advax-SM immunization generated a trend toward greater IFNγ⁺ CD4⁺ T cells at 4 dpi (FIG. 3B). By 8 dpi, similar increases in IFNγ⁺ CD4⁺ T cells were observed in all immunization groups. Validating the Th2-associated innate response, PreF/Alum immunization induced an increase in IL-4⁺ CD4⁺ T cells at 8 dpi (FIG. 3C) coupled with increases in IL-5⁺ and IL-13⁺ CD4⁺ T cells at 4 dpi that remained elevated through 8 dpi (FIGS. 3D-E). Alternatively, PreF/Advax-SM immunization generated greater numbers of CD8⁺ T cells at 4 and 8 dpi, though significance was lost by 8 dpi (FIG. 3F). Moreover, CD8⁺ T cells exhibited a cytotoxic phenotype in PreF/Advax-SM-vaccinated mice, with increased Granzyme B expression at both time points and increased IFNγ⁺ CD8⁺ T cells at 8 dpi (FIGS. 3G-H). Together, these data demonstrate that immunization of RSV-experienced young mice with PreF/Alum skews towards Th2 immunity as compared to PreF/Advax-SM, which favors a cytolytic CD8⁺ T cell response.

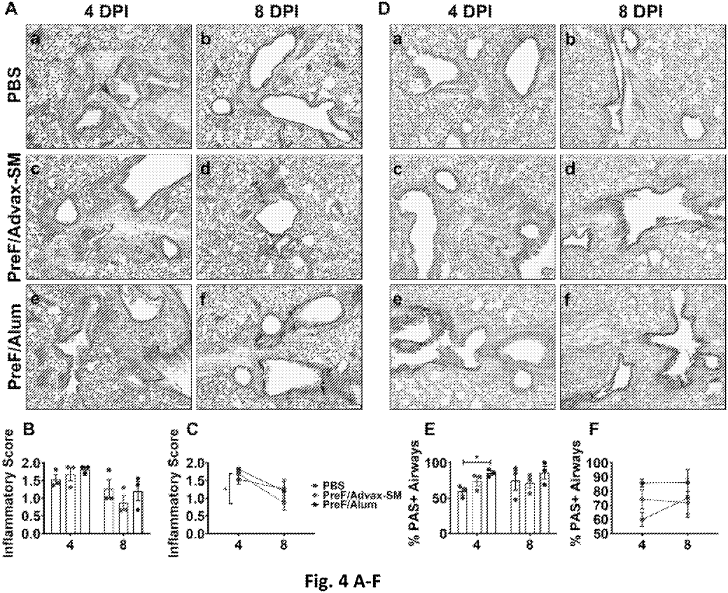

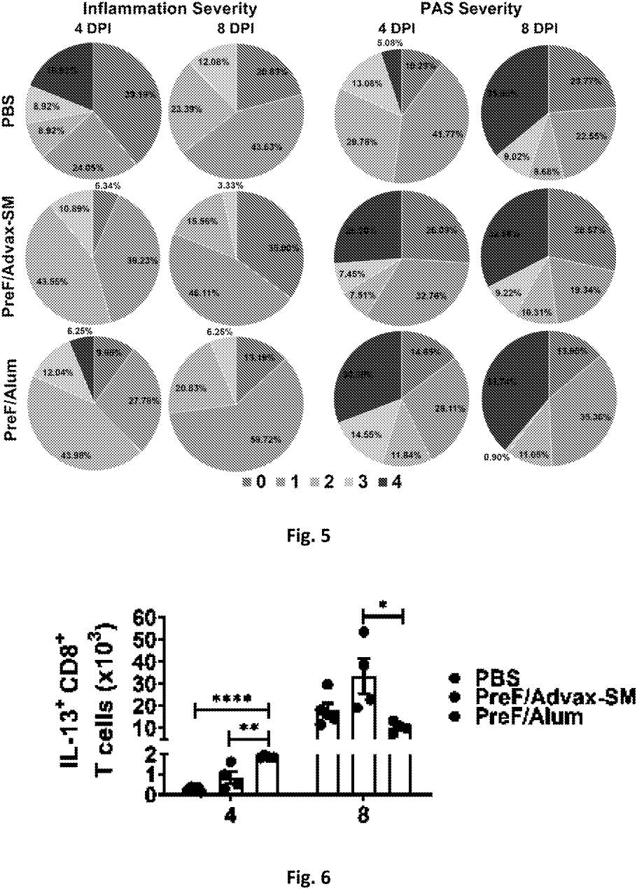

3.4. PreF/Alum-immunized young mice have increased airway mucus production following secondary RSV challenge. To evaluate whether the differential immune responses in PreF/Advax-SM- versus PreF/Alum-vaccinated animals corresponded with differences in pathology, lung sections were examined for inflammation and mucus production. At 4 dpi, all mice had peribronchial and perivascular inflammation but only PBS- and PreF/Alum-vaccinated animals had severity scores of 4 (FIG. 4A, a-f, FIG. 5). Overall, inflammation declined by 8 dpi (FIGS. 4B-C) but PreF/Advax-SM-vaccinated mice had the greatest reduction in inflammation (FIG. 4C).

Airway mucus production, another hallmark of RSV-mediated lung pathology, was lower in airways of PBS- versus PreF/Alum-immunized mice at 4 dpi (FIG. 4E), as determined by Periodic Acid-Schiff (PAS+) staining (FIG. 4D, a-f). However, the proportion of PAS+ airways trended up in the PBS group between 4 and 8 dpi, with > ⅓ of airways receiving severity scores of 4 (FIG. 4F & FIG. 5). Although the proportion of PAS+ airways in the PreF/Advax-SM mice trended lower than PreF/Alum at 4 dpi, the difference was not statistically significant. When evaluated over time, PAS+ airways increased in PBS mice, but remained low in the PreF/Advax-SM group (FIGS. 4E-F, FIG. 5). Alternatively, PreF/Alum-immunized mice maintained the highest proportion of PAS+ airways through 8 dpi (FIGS. 4E-F, FIG. 5). Overall, these data show similar levels of inflammation and mucus production across immunization groups, with notable early and persistently elevated levels of PAS+ airways with PreF/Alum immunization and faster resolution of inflammation in PreF/Advax-SM immunization mice.

### 4. Discussion

These results demonstrate that, despite prior infant RSV infection, PreF immunization of young mice can boost neutralizing antibody responses and produce discrete cellular immunity that is largely dependent on the vaccine adjuvant. Furthermore, our results suggest that high serum titers of vaccine-induced neutralizing antibodies and undetectable viral replication in the lungs do not guarantee protection from lung pathology in RSV-experienced immunized young mice.

RSV exposure occurs early in life, with nearly 70% of infants infected by the age of 1 [1]. These early-life responses to RSV are inefficient, characterized by an inability to produce neutralizing antibody and requiring multiple reinfections to develop short-term protective immunity [2, 9]. The recent stabilization of the prefusion conformation of RSV F protein (PreF) and its ability to generate potent neutralizing antibody when combined with both Th2-polarizing and Th1/Th2-balanced adjuvants has reinvigorated RSV vaccine development [10, 11]. Our results demonstrated that PreF-vaccination with either alum or Advax-SM adjuvants boosted RSV neutralizing antibody production in RSV-experienced young mice compared to PBS-immunized controls and conferred protection from reinfection.

RSV-infected human and murine neonates display an inability to mount strong IFNγ⁺ T cell responses [2, 4, 12], a characteristic that is associated with more severe acute disease [12] and exaggerated Th2-mediated pathology upon reinfection in mouse models [13, 14]. Therefore, in the context of pre-existing Th2-biased infant RSV immunity, vaccine formulations that promote IFNgamma-producing T cell responses may offer an improved safety and efficacy profile. Our results show that PreF immunization, when paired with the Th1/Th2-balanced adjuvant, Advax-SM, boosted neutralizing antibody production and induced an IFNγ⁺ cytotoxic CD8⁺ T cell response in RSV-experienced young mice. In contrast, PreF/Alum immunization generated robust Th2 immunity, characterized by increased airway eosinophils, IL-5⁺ and IL-13⁺ ILC2s, and Th2 CD4⁺ T cells, despite complete protection against viral replication. Contrary to previously published reports [13, 14], PBS controls in our study did not mount overt conventional Th2 immunity upon reinfection but was the only group to demonstrate an upward trajectory in the percentage of PAS+ airways between 4 and 8 dpi. This worsening over time suggests a possible delay in Th2 kinetics or unconventional sources of the mucus-inducing cytokine, IL-13.

While the elevated and sustained mucus hypersecretion in PreF/Alum-vaccinated mice was unsurprising given overwhelming Th2 immunity, mucus production in PreF/Advax-SM-vaccinated mice was unexpected. Although the evaluation of cellular immunity was limited to the airspace, PreF/Advax-SM-vaccinated mice had no appreciable type 2 response and no classic source of IL-13. Further T cell analysis demonstrated an increase in IL-13⁺ CD8⁺ T cells across all groups by 8 dpi with a notable increase in PreF/Advax-SM-vaccinated mice as compared to PreF/Alum-immunized mice (FIG. 6). This is consistent with the CD8⁺ Tc2 cell subset, which can express IL-13 and has been shown to play important roles during viral infection and allergic lung inflammation [15, 16]. Moreover, PBS controls generated a 61-fold increase in IL-13⁺ CD8⁺ T cells by 8 dpi (38.9- and 5.5-fold increases for PreF/Advax-SM and PreF/Alum, respectively), suggesting that delayed, unconventional sources of Th2 cytokines may contribute to lung pathology following repeated RSV infections. However, the increase in IFNγ⁺ CD8 T cells in PreF/Advax-SM-vaccinated animals at 8 dpi may have counteracted the increase in IL-13⁺ CD8 T cells, mitigating the increase in mucus production observed in PBS controls [17]. This is the first known report of CD8⁺ Tc2 in a young mouse model of RSV immunization and their role in RSV-mediated histopathology requires further study.

Most infants are exposed to RSV within their first year of life [1] but numerous exposures over multiple RSV seasons are required to achieve adult-like immunity [2]. Although adult RSV immune responses are associated with less severe disease than infants and children, the protection afforded through natural RSV infection in adulthood is short-lived [9]. Therefore, the goal of immunizing RSV-experienced young children is to generate Th1/Th2-balanced RSV immunity that provides long-lasting protection. Using a novel, clinically relevant murine model, we show for the first time that RSV Pre F immunization of RSV-experienced young mice can protect from reinfection. These data further demonstrate that adjuvants with greater Th1-skewing potential may ameliorate aspects of histopathology resulting from RSV memory generated during primary infant RSV infection.

### REFERENCES

Glezen WP, Taber LH, Frank AL, Kasel JA. Risk of primary infection and reinfection with respiratory syncytial virus. Am J Dis Child. 1986;140:543-6.

Green CA, Sande CJ, de Lara C, Thompson AJ, Silva-Reyes L, Napolitano F, et al. Humoral and cellular immunity to RSV in infants, children and adults. Vaccine. 2018;36:6183-90.

Bont L, Versteegh J, Swelsen WT, Heijnen CJ, Kavelaars A, Brus F, et al. Natural reinfection with respiratory syncytial virus does not boost virus-specific T-cell immunity. Pediatr Res. 2002;52:363-7.

Empey KM, Orend JG, Peebles RS, Jr., Egana L, Norris KA, Oury TD, et al. Stimulation of immature lung macrophages with intranasal interferon gamma in a novel neonatal mouse model of respiratory syncytial virus infection. PLoS One. 2012;7:e40499.

Graham BS, Perkins MD, Wright PF, Karzon DT. Primary respiratory syncytial virus infection in mice. J Med Virol. 1988;26:153-62.

Eichinger KM, Kosanovich JL, Empey KM. Localization of the T-cell response to RSV infection is altered in infant mice. Pediatr Pulmonol. 2018;53:145-53.

Eichinger KM, Egana L, Orend JG, Resetar E, Anderson KB, Patel R, et al. Alveolar macrophages support interferon gamma-mediated viral clearance in RSV-infected neonatal mice. Respir Res. 2015;16:122.

Perkins TN, Oczypok EA, Dutz RE, Donnell ML, Myerburg MM, Oury TD. The receptor for advanced glycation end products is a critical mediator of type 2 cytokine signaling in the lungs. J Allergy Clin Immunol. 2019;144:796-808 e12.

Hall CB, Walsh EE, Long CE, Schnabel KC. Immunity to and frequency of reinfection with respiratory syncytial virus. J Infect Dis. 1991;163:693-8.

McLellan JS, Chen M, Joyce MG, Sastry M, Stewart-Jones GB, Yang Y, et al. Structure-based design of a fusion glycoprotein vaccine for respiratory syncytial virus. Science. 2013;342:592-8.

Sastry M, Zhang B, Chen M, Joyce MG, Kong WP, Chuang GY, et al. Adjuvants and the vaccine response to the DS-Cav1-stabilized fusion glycoprotein of respiratory syncytial virus. PLoS One. 2017;12:e0186854.

Legg JP, Hussain IR, Warner JA, Johnston SL, Warner JO. Type 1 and type 2 cytokine imbalance in acute respiratory syncytial virus bronchiolitis. Am J Respir Crit Care Med. 2003;168:633-9.

Culley FJ, Pollott J, Openshaw PJ. Age at first viral infection determines the pattern of T cell-mediated disease during reinfection in adulthood. J Exp Med. 2002;196:1381-6.

Dakhama A, Park JW, Taube C, Joetham A, Balhorn A, Miyahara N, et al. The enhancement or prevention of airway hyperresponsiveness during reinfection with respiratory syncytial virus is critically dependent on the age at first infection and IL-13 production. J Immunol. 2005;175:1876-83.

Cerwenka A, Morgan TM, Harmsen AG, Dutton RW. Migration kinetics and final destination of type 1 and type 2 CD8 effector cells predict protection against pulmonary virus infection. J Exp Med. 1999;189:423-34.

Miyahara N, Takeda K, Kodama T, Joetham A, Taube C, Park JW, et al. Contribution of antigen-primed CD8+ T cells to the development of airway hyperresponsiveness and inflammation is associated with IL-13. J Immunol. 2004;172:2549-58.

Cohn L, Homer RJ, Niu N, Bottomly K. T helper 1 cells and interferon gamma regulate allergic airway inflammation and mucus production. J Exp Med. 1999;190:1309-18.

Eichinger et al., “Prefusion RSV F Immunization Elicits Th2-Mediated Lung Pathology in Mice When Formulated With a Th2 (but Not a Th1/Th2-Balanced) Adjuvant Despite Complete Viral Protection; Frontiers in Immunology; 2020; 11, 1673.

We claim:

1. A method of preventing or ameliorating respiratory syncytial virus (RSV) infection or eliciting a protective immune response against RSV infection in a juvenile subject, the method comprising administering to a juvenile subject an effective amount of:
a) an RSV F polypeptide stabilized in a prefusion conformation, and
b) a Th-balanced adjuvant, thereby preventing, ameliorating, or eliciting a protective immune response against respiratory syncytial virus (RSV) infection in the subject.

2. The method of claim 1, wherein the method results in the generation of a Th-balanced immune response in the subject.

3. The method of claim 1 or claim 2, wherein the method prevents, or ameliorates the development of, vaccine-enhanced respiratory disease (VERD) or eosinophilia in the subject.

4. The method of any of claims 1-3, wherein the method results in the elicitation of a neutralizing antibody response in the subject.

5. The method of any of claims 1-4, wherein the subject is a human subject.

6. The method of claim 5, wherein the subject is from bout 2 to about 15 years of age.

7. The method of claim 5, wherein the subject is from bout 3 to about 15 years of age.

8. The method of claim 5, wherein the subject is from bout 4 to about 15 years of age.

9. The method of claim 5, wherein the subject is from bout 5 to about 15 years of age.

10. The method of any of the preceding claims, wherein the RSV F polypeptide comprises one or more amino acid substitutions that partially or completely fill a cavity within the RSV F polypeptide.

11. The method of any of the preceding claims, wherein the RSV F polypeptide comprises an artificially-introduced disulfide bond.

12. The method of any of the preceding claims, wherein the RSV F polypeptide comprises one or more artificially-introduced dityrosine bonds.

13. The method of any of the preceding claims, wherein the RSV F polypeptide comprises one or more artificially-introduced “to-tyrosine” mutations.

14. The method of any of the preceding claims, wherein the RSV F polypeptide comprises one or more artificially-introduced “to-tyrosine” mutations and one or more artificially-introduced dityrosine bonds.

15. The method of any one of the preceding claims, wherein the Th-balanced adjuvant is, or comprises, CpG oligonucleotides, MPL, poly(I:C), poly(IC:LC), Freunds Complete Adjuvant, saponin, dQS21, an oil-in-water emulsion adjuvant, or Advax-SM.


SEQUENCE LISTING (9 sequences)

SEQ ID NO: 1 - protein, 513 aa, respiratory syncytial virus (RSV), subtype A
MELLILKANAITTILTAVTFCFASGQNITEEFYQSTCSAVSKGYLSALRTGWYTSVITIELSNIKKNKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELL

SEQ ID NO: 2 - protein, 513 aa, respiratory syncytial virus (RSV), subtype B
MELLIHRSSAIFLTLAINALYLTSSQNITEEFYQSTCSAVSRGYFSALRTGWYTSVITIELSNITETKCNGTDTKVKLIKQELDKYKNAVTELQLLMQNTPAANNRARREAPQHMNYTINTTKNLNVSISKKRKRRFLGFLLGVGSAIASGIAVSKVLHLEGEVNKIKNALLSTNKAVVSLSNGVSVLTSKVLDLKNYINNQLLPIVNQQSCRIFNIETVIEFQQKNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRKSDELL

SEQ ID NO: 3 - protein, 552 aa, Artificial Sequence
  Synthetic construct
MELLIHRSSAIFLTLAINALYLTSSQNITEEFYQSTCSAVSRGYFSALRTGWYTSVITIELSNITETKCNGTDTKVKLIKQELDKYKNAVTELQLLMQNTPAANNRARREAPQHMNYTINTTKNLNVSISKKRKRRFLGFLLGVGSAIASGIAVSKVLHLEGEVNKIKNALLSTNKAVVSLSNGVSVLTSKVLDLKNYINNQLLPIVNQQSCRIFNIETVIEFQQKNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRKSDELLSAIGGYIPEAPRDGQAYVRKDGEWVLLSTFLGGLVPRGS

SEQ ID NO: 4 - protein, 574 aa, Artificial Sequence
  Synthetic construct
MELLIHRSSAIFLTLAINALYLTSSQNITEEFYQSTCSAVSRGYFSALRTGWYTSVITIELSNITETKCNGTDTKVKLIKQELDKYKNAVTELQLLMQNTPAANNRARREAPQHMNYTINTTKNLNVSISKKRKRRFLGFLLGVGSAIASGIAVSKVLHLEGEVNKIKNALLSTNKAVVSLSNGVSVLTSKVLDLKNYINNQLLPIVNQQSCRIFNIETVIEFQQKNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRKSDELLHNVNTGKSTTNIMITTIIIVIIVVLLSLIAIGLLLYCKAKNTPVTLSKDQLSGINNIAFSK

SEQ ID NO: 5 - protein, 552 aa, Artificial Sequence
  Synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQYNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSAIGGYIPEAPRDGQAYVRKDGEWVLLSTFLGGLVPRGS

SEQ ID NO: 6 - protein, 552 aa, Artificial Sequence
  Synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSAIGGYIPEAPRDGQAYVRKDGEWVLLSTFLGGLVPRGS

SEQ ID NO: 7 - protein, 574 aa, Artificial Sequence
  Synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNTGKSTTNIMITTIIIVIIVVLLSLIAIGLLLYCKAKNTPVTLSKDQLSGINNIAFSK

SEQ ID NO: 8 - protein, 552 aa, Artificial Sequence
  Synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSAIGGYIPEAPRDGQAYVRKDGEWVLLSTFLGGLVPRGS

SEQ ID NO: 9 - protein, 574 aa, Artificial Sequence
  Synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNTGKSTTNIMITTIIIVIIVVLLSLIAIGLLLYCKAKNTPVTLSKDQLSGINNIAFSK